(19) United States
(12) Patent Application Publication
(10) Pub. No.: US 2018/0055342 A1
(43) Pub. Date: Mar. 1, 2018

(54) ENDOSCOPE AND OPTICAL TRANSMISSION MODULE

(71) Applicant: OLYMPUS CORPORATION, Tokyo (JP)

(72) Inventors: Youhei SAKAI, Ina-shi (JP); Hideharu MIYAHARA, Kamiina-gun (JP)

(73) Assignee: OLYMPUS CORPORATION, Tokyo (JP)

(21) Appl. No.: 15/800,345

(22) Filed: Nov. 1, 2017

Related U.S. Application Data
(63) Continuation of application No. PCT/JP2015/064186, filed on May 18, 2015.

(51) Int. Cl. A61B 1/00

(52) U.S. Cl. CPC A61B 1/00096; A61B 1/05; A61B 1/00013; A61B 1/00009

(57) ABSTRACT

An endoscope includes an insertion portion including an optical transmission module on a distal end portion, and an operation portion, the optical transmission module includes an optical fiber, a light emitting element, a holding member provided with a through-hole, a wiring board in which the light emitting element is bonded to a first main surface and the holding member is joined to the second main surface, and a transparent resin filling a space between a light emitting portion of the light emitting element and a distal end face of the optical fiber, and an entire periphery of a distal end portion of the optical fiber is tapered.

### CROSS REFERENCE TO RELATED APPLICATION

This application is a continuation application of PCT/JP2015/064186 filed on May 18, 2015, the entire contents of which are incorporated herein by this reference.

### BACKGROUND OF THE INVENTION

### 1. Field of the Invention

The present invention relates to an optical transmission module including an optical fiber configured to transmit an optical signal, an optical element, a holding member provided with a through-hole into which the optical fiber is inserted, and a wiring board provided with a hole portion to be an optical path of the optical signal, in which the holding member is joined to a first main surface and the optical element is mounted on a second main surface, and an endoscope including the optical transmission module on a distal end portion of an insertion portion.

### 2. Description of the Related Art

An endoscope includes an image pickup device such as a CCD at a distal end portion of an elongated flexible insertion portion. In recent years, use of an image pickup device with a high pixel number in an endoscope has been examined In a case of using an image pickup device with a high pixel number, a signal amount transmitted from the image pickup device to a signal processing apparatus (processor) increases so that optical signal transmission through a thin optical fiber by an optical signal is desirable instead of electric signal transmission through metal wiring by an electric signal. For the optical signal transmission, an E/O optical transmission module (electro-optical converter) configured to convert the electric signal to the optical signal and an O/E optical transmission module (opto-electrical converter) configured to convert the optical signal to the electric signal are used.

For the optical transmission module, accurate positioning and fixation are important in order to optically couple an optical element and an optical fiber configured to transmit the optical signal. In order to accurately and easily position the optical element and the optical fiber, for the optical transmission module, a holding member (ferrule) including a through-hole disposed on a wiring board on which the optical element is mounted. By inserting the optical fiber to the through-hole of the holding member, a horizontal direction of the optical element and the optical fiber can be easily positioned. For accurate positioning, a diameter of the through-hole is set slightly larger than an outer diameter of the optical fiber.

In order to fix the optical element and the optical fiber, before inserting the optical fiber to the through-hole, an uncured transparent resin in a liquid state is injected to the through-hole. Then, the optical fiber is inserted into the through-hole so as to extrude the transparent resin, and the transparent resin is cured. Thus, the optical fiber and the optical element are tightly fixed. In addition, to an optical path between the optical fiber and the optical element, the transparent resin is filled.

Japanese Patent Application Laid-Open Publication No. 2012-198451 discloses a holding member with an adhesive agent housing portion configured to house excess transparent resin formed in a through-hole of the holding member.

### SUMMARY OF THE INVENTION

An endoscope of an embodiment includes: an insertion portion including an optical transmission module at a distal end portion where an image pickup device is disposed; and an operation portion extended on a proximal end portion side of the insertion portion, and the optical transmission module includes an optical fiber inserted through the insertion portion and configured to transmit an optical signal, an optical element, on a surface of which an optical element portion and an external electrode are disposed, the optical element portion being configured to emit the optical signal or receive the optical signal that is made incident, a holding member provided with a through-hole into which the optical fiber is inserted, the holding member being formed of a transparent material that transmits ultraviolet rays, a wiring board provided with a hole portion to be an optical path of the optical signal, in which a bond electrode disposed on a first main surface and the external electrode of the optical element are bonded and the holding member is joined to a second main surface, and an ultraviolet curing type transparent resin filling a space between the optical element portion of the optical element and a distal end face of the optical fiber. An entire periphery of a distal end portion of the optical fiber is tapered, a diameter of the through-hole of the holding member is smaller than an outer diameter of a non-worked portion of the optical fiber, and the transparent resin enters the space formed by taper working of the optical fiber.

An optical transmission module of another embodiment includes: an optical fiber configured to transmit an optical signal; an optical element, on a surface of which an optical element portion and an external electrode are disposed, the optical element portion being configured to emit the optical signal or receive the optical signal that is made incident; a holding member provided with a through-hole into which the optical fiber is inserted; a wiring board provided with a hole portion to be an optical path of the optical signal, in which a bond electrode disposed on a first main surface and the external electrode of the optical element are bonded and the holding member is joined to a second main surface; and a transparent resin filling a space between the optical element portion of the optical element and a distal end face of the optical fiber. A part of a distal end portion of the optical fiber is removed, and the transparent resin enters the space formed by the removal of the optical fiber.

### BRIEF DESCRIPTION OF THE DRAWINGS

FIG. 1 is a perspective view of an endoscope in a first embodiment;

FIG. 2A is a sectional view of an optical transmission module in the first embodiment;

FIG. 2B is a top view of the optical transmission module in the first embodiment;

FIG. 3 is a perspective view of an optical fiber of the optical transmission module in the first embodiment;

FIG. 4A is a bottom view of the optical fiber of the optical transmission module in the first embodiment;

FIG. 4B is a bottom view of the optical fiber of the optical transmission module in a modification of the first embodiment;

FIG. 4C is a bottom view of the optical fiber of the optical transmission module in the modification of the first embodiment;

FIG. 5 is a sectional view of the optical transmission module in a second embodiment;

FIG. 6A is a sectional view of the optical transmission module in a third embodiment;

FIG. 6B is a top view of the optical transmission module in the third embodiment;

FIG. 7A is a sectional view of the optical fiber of the optical transmission module in the third embodiment;

FIG. 7B is a sectional view of the optical fiber of the optical transmission module in a modification of the third embodiment;

FIG. 7C is a sectional view of the optical fiber of the optical transmission module in the modification of the third embodiment;

FIG. 7D is a sectional view of the optical fiber of the optical transmission module in the modification of the third embodiment;

FIG. 7E is a sectional view of the optical fiber of the optical transmission module in the modification of the third embodiment; and

FIG. 8 is a sectional view of the optical transmission module in a fourth embodiment.

### DETAILED DESCRIPTION OF THE PREFERRED EMBODIMENT(S)

### First Embodiment

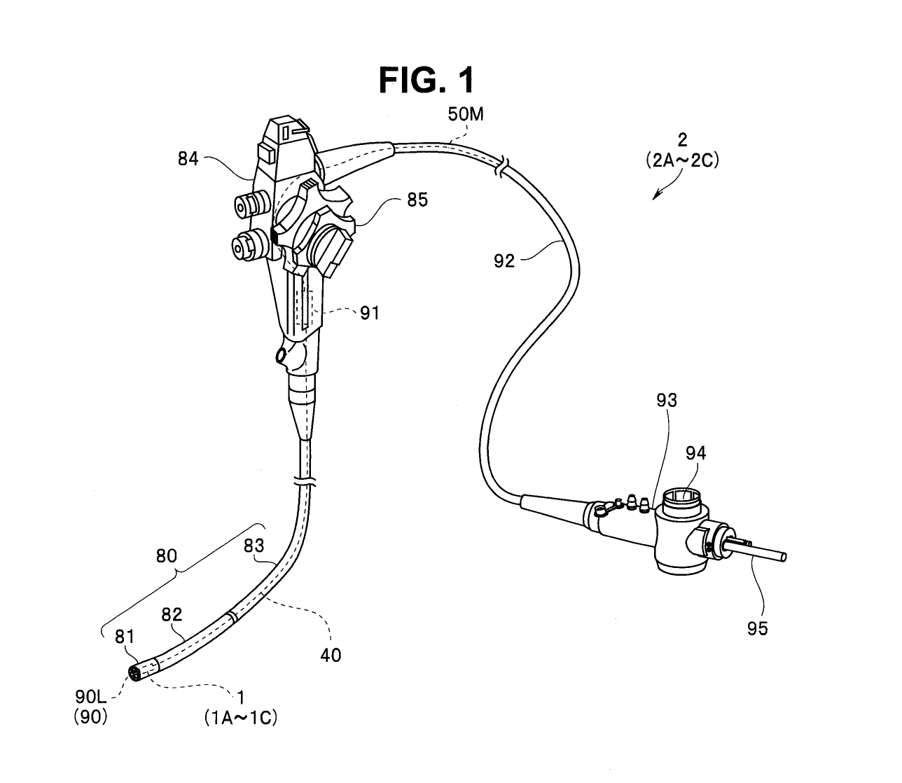

As illustrated in FIG. 1, an endoscope 2 in the present embodiment includes an insertion portion 80, an operation portion 84 disposed on a proximal end portion side of the insertion portion 80, a universal cord 92 extended from the operation portion 84, and a connector 93 disposed on the proximal end portion side of the universal cord 92.

For the insertion portion 80, a rigid distal end portion 81, a bending portion 82 for changing a direction of the distal end portion 81, and an elongated flexible portion 83 are connected in order.

In the distal end portion 81, an image pickup optical unit 90L, an image pickup device 90, and an optical transmission module 1 which is an E/O module configured to convert an image pickup signal (electric signal) from the image pickup device 90 to an optical signal are disposed. The image pickup device 90 is a CMOS (complementary metal oxide semiconductor) image sensor or a CCD (charge coupled device) or the like.

In the operation portion 84, an angle knob 85 configured to operate the bending portion 82 is disposed, and also an O/E module 91 which is an optical transmission module configured to convert the optical signal to the electric signal is disposed. The connector 93 includes an electric connector portion 94 connected with a processor (not illustrated), and a light guide connection portion 95 connected with a light source. The light guide connection portion 95 is connected with an optical fiber bundle which guides illumination light to the rigid distal end portion 81. Note that, in the connector 93, the electric connector portion 94 and the light guide connection portion 95 may be united.

In the endoscope 2, the image pickup signal is converted to the optical signal in the optical transmission module 1 which is the E/O module disposed in the distal end portion 81 or the like, and is transmitted through a thin optical fiber 40 inserted in the insertion portion 80 to the operation portion 84. Then, the optical signal is converted to the electric signal again by the O/E module 91 disposed in the operation portion 84, and is transmitted through metal wiring 50M inserted in the universal cord 92 to the electric connector portion 94. That is, the signal is transmitted through the optical fiber 40 inside the insertion portion 80 of a narrow diameter, and the signal is transmitted through the metal wiring 50M thicker than the optical fiber 40 inside the universal cord 92 which is not inserted into a body and has little limitation on an outer diameter.

Note that, in a case where the O/E module 91 is arranged in a vicinity of the electric connector portion 94, the optical fiber 40 may be inserted in the universal cord 92 to the vicinity of the electric connector portion 94. In addition, in the case where the O/E module 91 is disposed in the processor, the optical fiber 40 may be inserted up to the connector 93.

In the endoscope 2, since optical signal transmission through the thin optical fiber 40 by the optical signal is performed instead of electric signal transmission, the insertion portion 80 is thin and less invasive.

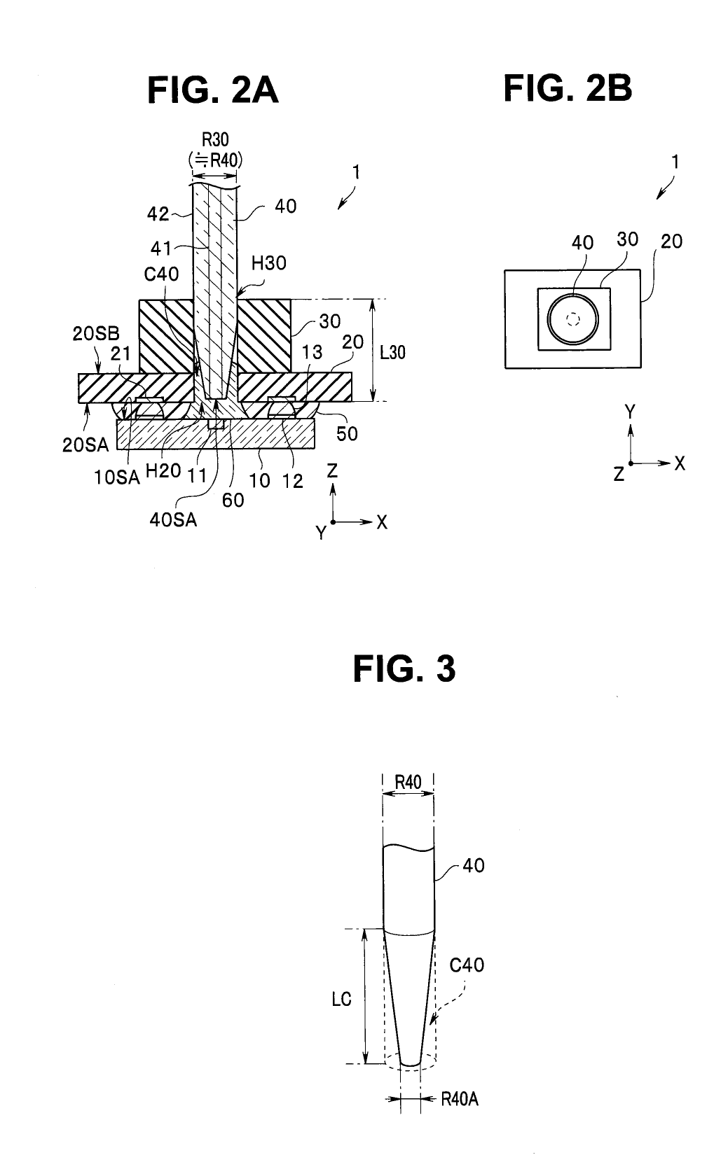

As illustrated in FIG. 2A and FIG. 2B, the optical transmission module 1 in the present embodiment includes an optical element 10 which is a light emitting element, a wiring board 20, a holding member (also referred to as a ferrule) 30, and the optical fiber 40 inserted in the insertion portion 80. In the optical transmission module 1, the optical element 10, the wiring board 20 and the holding member 30 are arranged side by side in a thickness direction (Z direction) of the optical element 10.

Note that, in following description, the drawings based on the individual embodiments are schematic, it should be noted that a relation between a thickness and a width of individual parts and a ratio of the thicknesses of the respective parts and relative angles or the like are different from the actual ones, and even between the drawings, a part where the relation of mutual dimensions or the ratio is different is sometimes included. In addition, a direction of increasing a value of a Z axis is sometimes referred to as “up”.

The optical element 10 is a surface light emitting laser chip in which a light emitting portion 11 that is a light element portion configured to output light of the optical signal is formed on a light emitting surface 10SA that is a front surface. For example, an ultra-small optical element 10, a planar view dimension of which is 250 μm×300 μm, includes the light emitting portion 11, a diameter of which is 20 μm, and an external electrode 12 configured to supply a drive signal to the light emitting portion 11 on the light emitting surface 10SA.

On the other hand, for example, the optical fiber 40 includes a core portion 41 of a 50 μm diameter configured to transmit the light, and a clad portion 42 of a 125 μm diameter configured to cover an outer peripheral surface of the core portion 41. The core portion 41 is formed of glass, a refractive index of which is slightly smaller, about 0.2% to 0.3% for example, than the refractive index of the clad portion 42.

The holding member 30 of an approximately parallelepiped shape joined on the optical element 10 is provided with a through-hole H30 into which a distal end portion of the optical fiber 40 is inserted. By inserting and fitting the optical fiber 40 into the through-hole H30, the light emitting portion 11 of the optical element 10 and the optical fiber 40 are positioned. The diameter (inner diameter) of the through-hole H30 may be, other than a columnar shape, a prism shape such as a quadrangular prism or a hexagonal prism as long as the optical fiber 40 can be held by a wall surface of the through-hole H30. A material of the holding member 30 is ceramic, silicon, glass or a metal member such as SUS or the like. Note that the holding member 30 may be in an approximately columnar shape or an approximately conical shape or the like.

As already described, at the holding member 30, the columnar through-hole H30, a diameter R30 of which is almost same as an outer diameter R40 of the optical fiber 40 to be inserted, is formed. Here, “almost same” means that both diameters are practically a “same” size such that an outer peripheral surface of the optical fiber 40 and the wall surface of the through-hole H30 are brought into contact and turned to a fitted state. For example, the diameter R30 of the through-hole H30 is manufactured to be larger than the outer diameter R40 of the optical fiber 40 by only 1 μm to 5 μm.

The planar wiring board 20 including a first main surface 20SA and a second main surface 20SB is provided with a hole portion H20 to be an optical path. A bond electrode 21 disposed on the first main surface 20SA of the wiring board 20 and the external electrode 12 of the optical element 10 are bonded through a bump 13. That is, the optical element 10 is flip-chip mounted on the wiring board 20 in a state where the light emitting portion 11 is arranged at a position opposing the hole portion H20 of the wiring board 20. For example, a stud gold bump 13 is ultrasonically bonded with the bond electrode 21 of the wiring board 20. Therefore, between the light emitting portion 11 of the optical element 10 and the first main surface 20SA of the wiring board 20, a gap corresponding to a height of the bump 13 is provided.

For a base of the wiring board 20, an FPC substrate, a ceramic substrate, a glass epoxy substrate, a glass substrate, a silicon substrate, or the like is used.

Note that solder paste or the like may be printed on the wiring board 20 to attain a bump, and the optical element 10 may be arranged at a predetermined position and then mounted by melting solder by reflow or the like. Note that the wiring board 20 may include a processing circuit for converting the electric signal transmitted from the image pickup device 90 to the drive signal of the optical element 10 or the like.

On the second main surface 20SB of the wiring board 20, the holding member 30 is joined with an adhesive agent (not illustrated) in the state where the through-hole H30 is arranged at a position opposing the hole portion H20.

In a manufacturing process of the optical transmission module 1, for example, after the optical element 10 is flip-chip mounted on the first main surface 20SA of the wiring board 20, the holding member 30 is joined to the second main surface 20SB of the wiring board 20. Note that the optical element 10 may be mounted on the wiring board 20 to which the holding member 30 is joined.

A bond portion of the bond electrode 21 and the external electrode 12 is sealed by injection of a sealing resin 50 such as an underfill material or a sidefill material. For the sealing resin 50, a resin excellent in moisture resistance such as an epoxy resin or a silicone resin is used. Note that the sealing resin 50 is not an essential component, and the bond portion of the bond electrode 21 and the external electrode 12 may be bonded with a transparent resin 60.

In order to fix the optical fiber 40, an appropriate amount of the non-cured transparent resin 60 in a liquid state is injected to the through-hole H30 of the holding member 30 using a dispenser or the like. In order to more surely fill the space between the light emitting portion 11 and a distal end face 40SA with the transparent resin 60, a lot of the transparent resin 60 is injected. Then, the optical fiber 40 is inserted into the through-hole H30. While an insertion amount of the optical fiber 40 is appropriately adjusted, the optical fiber 40 may be completely inserted through the through-hole H30 of the holding member 30 or may be inserted even to the hole portion H20 of the wiring board 20 further.

Therefore, the space between the light emitting portion 11 of the optical element 10 and the distal end face 40SA of the optical fiber 40 is filled with the transparent resin 60. The transparent resin 60 is formed on an ultraviolet curing type resin. Then, by irradiation with ultraviolet rays after the optical fiber 40 is inserted, curing treatment is performed.

In order to efficiently irradiate the transparent resin 60 injected to the through-hole H30 with the ultraviolet rays, it is preferable that the holding member 30 is formed of glass that transmits the ultraviolet rays. Note that it is easy to manufacture the optical transmission module 1 even with the glass holding member 30.

A thermosetting type resin can be also used as the transparent resin 60, but the optical element 10 may be deteriorated by heating treatment. Therefore, it is preferable to use the holding member 30 formed of the glass that transmits the ultraviolet rays and the transparent resin 60 of the ultraviolet curing type.

For the transparent resin 60, a curable resin, the refractive index of which is almost same as the refractive index of the core portion 41 of the optical fiber 40, is used. Since the refractive index of the core portion 41 is about 1.4 to 1.6, for example a silicone-based resin, an epoxy-based resin or an acrylic resin, the refractive index of which after being cured is about 1.4 to 1.6, is used for the transparent resin 60. The transparent resin 60 with a refractive index which is almost the same as the refractive index of the core portion 41 causes a small coupling loss on an interface.

Since the transparent resin 60 serves as the optical path, the transparent resin 60 is selected from resins of a high light transmittance so as not to attenuate light emitted by the light emitting portion 11. Note that, in the case where the light emitting portion 11 generates infrared light, the transparent resin 60 may be opaque in a visible light region, that is, a transmittance of the transparent resin 60 may be low, as long as a light transmittance in an infrared region is high.

As illustrated in FIG. 3, in the optical transmission module 1, a part of a length LC at the distal end portion of the optical fiber 40 is removed gradually over an entire periphery. Then, the transparent resin 60 extruded by insertion of the optical fiber 40 enters a space C40 between the wall surface of the through-hole and an outer peripheral surface formed by removal of the optical fiber 40.

That is, the entire periphery of the optical fiber 40 is circularly tapered so that the distal end portion is tapered. The outer diameter of the tapered distal end portion gradually becomes small toward a distal end, and an outer diameter R40A of the distal end face 40SA is smaller than the outer diameter of a non-worked portion (the outer diameter of the clad portion 42) R40.

As already described, in the non-tapered optical fiber, the outer diameter R40 and the diameter R30 of the through-hole H30 are almost the same. Therefore, when the optical fiber is inserted, air inside the through-hole H30 is not easily expelled, and air bubbles may be caught in the transparent resin and are left in the optical path.

In contrast, since the optical fiber 40 is tapered at the distal end portion, when the optical fiber 40 is inserted into the through-hole H30, the air is promptly expelled through the space (gap) where the removal is performed on the outer peripheral surface of the optical fiber 40, and the excessively injected transparent resin 60 enters the space where the removal is performed.

Therefore, the air bubbles do not remain in the transparent resin 60 between the light emitting portion 11 of the optical element 10 and the distal end face 40SA of the optical fiber 40. In addition, there is no risk that the optical element 10 is detached from the wiring board 20 by a pressure of inserting the optical fiber 40.

Since the air bubbles do not remain in the transparent resin 60 in the optical path, in the optical transmission module 1, the coupling efficiency of the optical fiber 40 and the optical element 10 is high.

Note that it is sufficient when the outer diameter R40A of the distal end face 40SA of the optical fiber 40 is equal to or larger than an outer dimension of the light emitting portion 11, and it is preferable that the outer diameter R40A is 1.5 times or more of the diameter of the light emitting portion 11 and is equal to or lower than 90% of the outer diameter of the non-worked portion (the outer diameter of the clad portion) R40, since a light quantity does not decline and the air bubbles do not remain.

In addition, the outer diameter of the optical fiber 40 is linearly reduced toward the distal end by taper working, but may be reduced in a curved shape.

Note that, in the optical transmission module 1, the length LC of a tapered portion is shorter than a length L30 which is a total of a thickness of the holding member 30 and a thickness of the wiring board 20. Therefore, when the optical fiber 40 is inserted into the through-hole H30, the tapered portion is housed inside the through-hole H30.

### Modification of First Embodiment

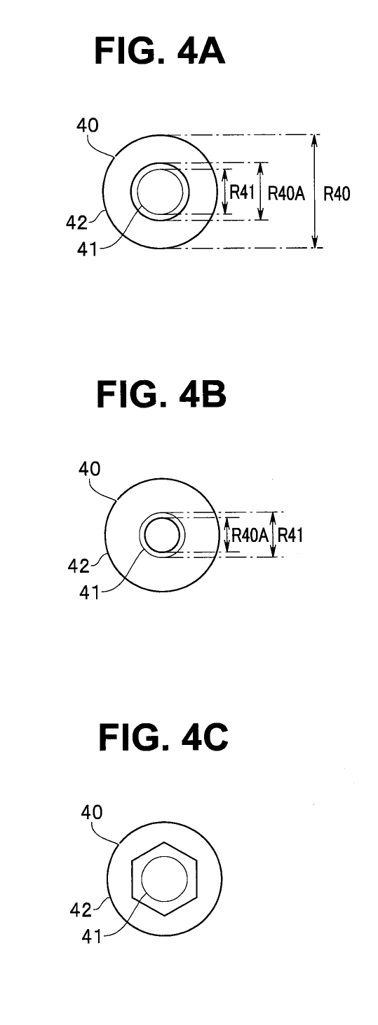

As illustrated in FIG. 4A, the outer diameter R40A of the distal end face of the tapered portion is larger than an outer diameter R41 of the core portion 41 in the optical transmission module 1. That is, the core portion 41 is not tapered. However, as illustrated in FIG. 4B, taper working may be performed to the core portion 41. That is, the outer diameter R40A of the distal end face of the tapered portion may be smaller than the outer diameter R41 of the core portion 41. Note that, in this case, it is preferable to use a material of the refractive index almost the same as the refractive index of the clad portion 42 as the transparent resin 60 for optical transmission efficiency improvement.

In addition, in the optical transmission module 1, a sectional shape of the tapered portion is circular. In contrast, as illustrated in FIG. 4C, taper working may be performed into a polygon.

### Second Embodiment

An optical transmission module 1A in the second embodiment and an endoscope 2A including the optical transmission module 1A are similar to the optical transmission module 1 and the endoscope 2 and have the same effects so that same signs are attached to the components of same functions and the description is omitted.

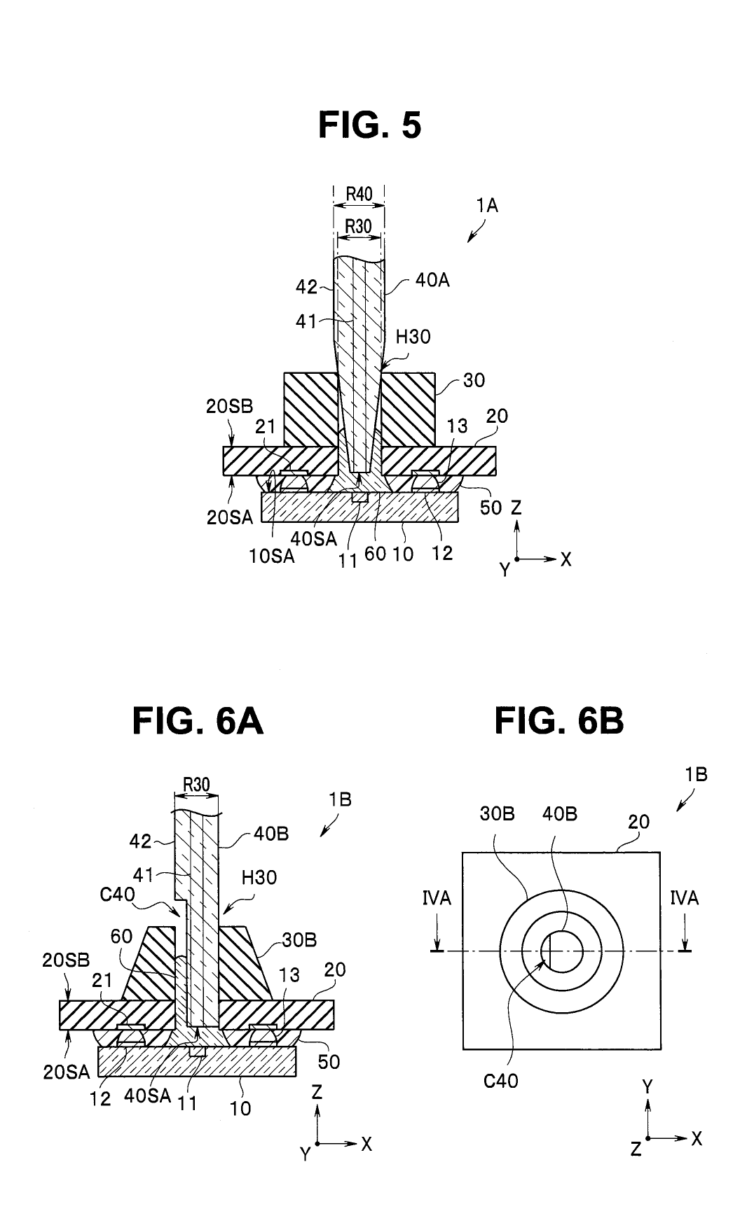

As illustrated in FIG. 5, in the optical transmission module 1A, the outer diameter R40A of the distal end face 40SA of the optical fiber 40A is smaller than the diameter R30 of the through-hole H30 of the holding member 30, and the outer diameter R40 of the non-worked portion is larger than the diameter R30 of the through-hole H30.

Therefore, an outer surface at an upper part of the tapered portion of the optical fiber 40A is in contact with an opening of the through-hole H30 on an upper surface of a holding member 30B. That is, a distance between the distal end face 40SA of the optical fiber 40A and the light emitting portion 11 of the optical element 10 is defined by a shape of the tapered portion.

In the optical transmission module 1A, by defining the shape of the tapered portion, the distance between the distal end face 40SA of the optical fiber 40A and the light emitting portion 11 of the optical element 10 can be accurately positioned. Therefore, in the optical transmission module 1A, the coupling efficiency of the optical fiber 40 and the optical element 10 is more stable.

### Third Embodiment

An optical transmission module 1B in the third embodiment and an endoscope 2B including the optical transmission module 1B are similar to the optical transmission module 1 and the endoscope 2 and have the same effects so that the same signs are attached to the components of the same functions and the description is omitted.

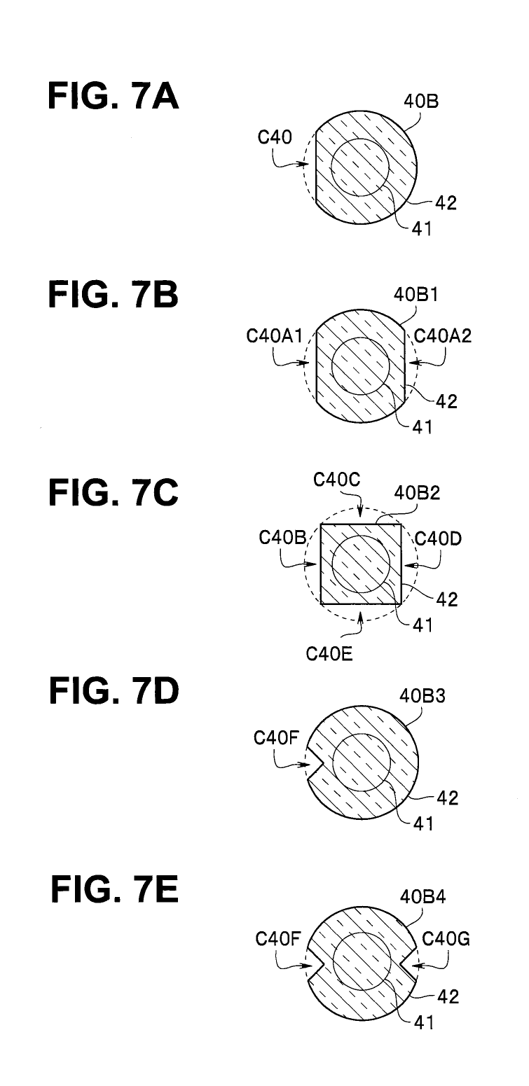

As illustrated in FIG. 6A, FIG. 6B and FIG. 7A, in the optical transmission module 1B, the holding member 30B is roughly conical with a trapezoidal cross section. In addition, for the optical fiber 40B, the distal end portion is cut out along a long axis, and the cross section is roughly D-shaped. That is, for the optical fiber 40B, a part of the distal end portion is removed.

A cutout portion C40 of the optical fiber 40B is formed to a part slightly above the opening of the through-hole H30 in the state of being inserted into the through-hole H30. When the optical fiber 40B is inserted into the through-hole H30, the air is expelled through the cutout portion C40, and also the transparent resin 60 intrudes the cutout portion C40. Therefore, the air bubbles do not remain in the transparent resin 60 between the light emitting portion 11 of the optical element 10 and the distal end face 40SA of the optical fiber 40B, and the optical fiber 40B is surely fixed to the holding member 30B.

Since the air bubbles do not remain in the transparent resin 60 in the optical path, in the optical transmission module 1B, the coupling efficiency of the optical fiber 40B and the optical element 10 is high. In addition, since an adhesive agent housing portion is not formed in the holding member 30B, the outer diameter of the optical transmission module 1B, that is, the outer dimension in an XY plane direction, is small. In the endoscope 2B including the optical transmission module 1B on the distal end portion 81, the distal end portion 81 has a small diameter and is lowly invasive.

### Modification of Third Embodiment

It is sufficient when the cutout portion formed in the optical fiber includes a sectional area necessary and sufficient for promptly expel the air when the optical fiber 40B is inserted into the through-hole H30.

For example, in an optical fiber 40B1 illustrated in FIG. 7B, two cutout portions C40A1 and C40A2 in the almost same shape are formed. In the optical fiber 40B1, even by the two shallow cutout portions C40A1 and C40A2 that are worked more easily than the cutout portion C40 of the optical fiber 40B, the same effects can be obtained as long as a total sectional area is almost the same as the sectional area of the cutout portion C40.

The outer peripheral surface of an optical fiber 40B2 illustrated in FIG. 7C is formed of four planes by four cutout portions C40B to C40E in almost the same shape. It can be also considered that the entire periphery of the optical fiber 40B2 is tapered.

In addition, for a cutout portion C40F of an optical fiber 40B3 illustrated in FIG. 7D, the cross section is in a V shape. The optical fiber 40B3 is worked more easily than the optical fiber 40B. In the optical fiber 40B4 illustrated in FIG. 7E, two cutout portions C40F and C40G, the cross sections of which are in almost the same V shape, are formed.

All the optical transmission modules in the modifications including the optical fibers 40B1 to 40B4 have the same effects as the effects of the optical transmission module 1A.

### Fourth Embodiment

An optical transmission module 1C in the fourth embodiment and an endoscope 2C including the optical transmission module 1C are similar to the optical transmission module 1B and the endoscope 2B or the like and have the same effects so that the same signs are attached to the components of the same functions and the description is omitted.

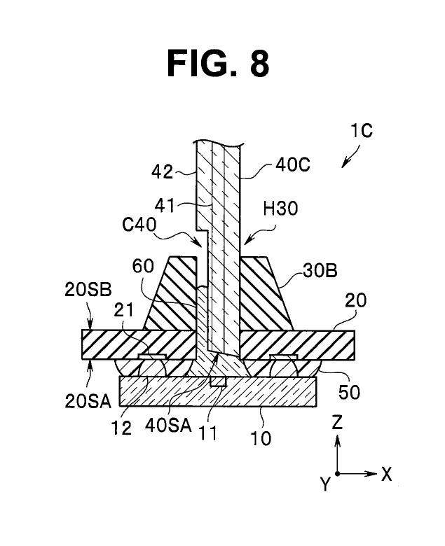

As illustrated in FIG. 8, the distal end portion of the optical fiber 40C in the optical transmission module 1C is cut out, and the cross section is roughly D-shaped. Further, in the optical fiber 40C, the distal end face 40SA is not vertical to a long axis direction, but is an inclined surface.

Since the distal end face 40SA is inclined, the air and the transparent resin 60 flow to the upper part without staying on the distal end face 40SA. Therefore, the optical transmission module 1C has the effects of the optical transmission module 1B or the like, and further, since the air bubbles do not easily remain in the transparent resin 60 in the optical path further, the coupling efficiency of the optical fiber 40B and the optical element 10 is higher.

Note that it is preferable that an inclination angle of the distal end face 40SA is equal to or larger than 1 degree and equal to or smaller than 10 degrees to a plane vertical to the long axis direction, and it is more preferable that the inclination angle is equal to or larger than 2 degrees and equal to or smaller than 4 degrees. The air bubbles do not remain at or above a lower limit of the range described above, and the light is easily made incident on the optical fiber 40B at or under a condition of the range. In addition, since multiple reflection does not easily occur between the distal end face 40SA and the light emitting surface 10SA, the coupling efficiency (transmission efficiency) is high.

Furthermore, also in the optical transmission modules 1 and 1A, by turning the distal end face 40SA of the optical fibers 40 and 40A to the inclined surface like the optical fiber 40C, the effects similar to the effects of the optical transmission module 1C can be obtained.

Note that the optical transmission module including the light emitting element as the optical element 10 or the like is described as an example above. However, it is needless to say that, even when the optical element is a light receiving element such as a photodiode, the similar effects are provided as long as the similar configuration is provided.

An O/E optical transmission module disposed at the distal end portion of the endoscope transmits a clock signal inputted to the image pickup device as the optical signal, for example. For the endoscope that transmits the clock signal through the thin optical fiber 40, the insertion portion 80 is thin and less invasive.

As described above, the optical transmission module of another embodiment of the present invention includes: an optical fiber configured to transmit an optical signal; a light receiving element, on a light receiving surface that is a surface of which a light receiving portion and an external electrode are disposed, the light receiving portion being where the optical signal is made incident; a holding member provided with a through-hole into which the optical fiber is inserted; a wiring board provided with a hole portion to be an optical path of the optical signal, in which a bond electrode disposed on a first main surface and the external electrode of the light receiving element are bonded and the holding member is joined to a second main surface; and a transparent resin filling a space between the light receiving portion of the light receiving element and a distal end face of the optical fiber. A part of a distal end portion of the optical fiber is removed, and the transparent resin enters the space formed by the removal of the optical fiber. In the optical transmission module, since the air bubbles do not easily remain in the transparent resin in the optical path, the coupling efficiency of the optical fiber and the light receiving element is high. In addition, the endoscope including the optical transmission module at the distal end portion of an insertion portion has a small diameter.

The present invention is not limited to the embodiments and the modifications or the like described above, and various changes, combinations and applications are possible without departing from the spirit of the invention.

What is claimed is:

1. An endoscope comprising:
an insertion portion including an optical transmission module at a distal end portion where an image pickup device is disposed; and
an operation portion extended on a proximal end portion side of the insertion portion,
wherein the optical transmission module includes
an optical fiber inserted through the insertion portion and configured to transmit an optical signal,
an optical element, on a surface of which an optical element portion and an external electrode are disposed, the optical element portion being configured to emit the optical signal or receive the optical signal that is made incident,
a holding member provided with a through-hole into which the optical fiber is inserted, the holding member being formed of a transparent material that transmits ultraviolet rays,
a wiring board provided with a hole portion to be an optical path of the optical signal, in which a bond electrode disposed on a first main surface and the external electrode of the optical element are bonded and the holding member is joined to a second main surface, and
an ultraviolet curing type transparent resin filling a space between the optical element portion of the optical element and a distal end face of the optical fiber,
an entire periphery of a distal end portion of the optical fiber is tapered,
a diameter of the through-hole of the holding member is smaller than an outer diameter of a non-worked portion of the optical fiber, and
the transparent resin enters the space formed by taper working of the optical fiber.

2. An optical transmission module comprising:
an optical fiber configured to transmit an optical signal;
an optical element, on a surface of which an optical element portion and an external electrode are disposed, the optical element portion being configured to emit the optical signal or receive the optical signal that is made incident;
a holding member provided with a through-hole into which the optical fiber is inserted;
a wiring board provided with a hole portion to be an optical path of the optical signal, in which a bond electrode disposed on a first main surface and the external electrode of the optical element are bonded and the holding member is joined to a second main surface; and
a transparent resin filling a space between the optical element portion of the optical element and a distal end face of the optical fiber,
wherein a part of a distal end portion of the optical fiber is removed, and
the transparent resin enters the space formed by the removal of the optical fiber.

3. The optical transmission module according to claim 2, wherein an entire periphery of the distal end portion of the optical fiber is tapered.

4. The optical transmission module according to claim 3, wherein a diameter of the through-hole of the holding member is smaller than an outer diameter of a non-worked portion of the optical fiber.

5. The optical transmission module according to claim 2, wherein the distal end portion of the optical fiber is cut out.

6. The optical transmission module according to claim 2, wherein the distal end face of the optical fiber is an inclined surface.

7. The optical transmission module according to claim 2,
wherein the holding member is formed of a transparent material that transmits ultraviolet rays, and
the transparent resin is an ultraviolet curing type.
